(19) United States
(12) Patent Application Publication
(10) Pub. No.: US 2021/0330186 A1
(43) Pub. Date: Oct. 28, 2021

(54) PORTABLE SCREENING DEVICES AND SYSTEMS FOR REMOTE OPTHALMIC DIAGNOSTICS

(71) Applicants: The Regents of the University of California, Oakland, CA (US); Melles Research Foundation USA, Inc., San Diego, CA (US)

(72) Inventors: Frank TALKE, Rancho Santa Fe, CA (US); Gerrit Reinold Jacob MELLES, Rotterdam (NL); Alex PHAN, San Diego, CA (US); Phuong TRUONG, El Monte, CA (US); Buu TRUONG, Norwalk, CA (US); Nicolas WILLIAMS, San Diego, CA (US)

(73) Assignees: The Regents of the University of California, Oakland, CA (US); Melles Research Foundation USA, Inc., San Diego, CA (US)

(21) Appl. No.: 17/285,946

(22) PCT Filed: Oct. 23, 2019
(86) PCT No.: PCT/US2019/057618

§ 371 Date: Apr. 16, 2021

Related U.S. Application Data
(60) Provisional application No. 62/843,059, filed on May 3, 2019.

(30) Foreign Application Priority Data
Oct. 24, 2018 (NL) 2021870

(51) Int. Cl. A61B 3/18; A61B 3/00; A61B 3/14; A61B 5/00; A61B 3/028; A61B 3/16; A61B 3/117; A61B 8/06; A61B 8/08

(52) U.S. Cl. CPC A61B 3/18; A61B 3/0008; A61B 3/14; A61B 5/0022; A61B 8/488; A61B 3/165; A61B 3/0083; A61B 3/1176; A61B 8/06; A61B 3/028

(57) ABSTRACT

A device provides remote ophthalmic examinations and includes one, plural or all of the following capabilities: slit lamp examinations, visual acuity examinations, fundoscopy, and eye pressure assessment. Preferred devices enable a patient to self-align or adjust device themselves without the help of a professional, collect and store data and can transmit data when a connection is available to a professional or an analysis system, such as a machine learning system, for evaluation.

### PRIORITY CLAIM AND REFERENCE TO RELATED APPLICATION

The application claims priority under 35 U.S.C. § 119 and all applicable statutes and treaties from prior Netherlands Patent Application serial number N2021870, which was filed Oct. 24, 2018 and from prior U.S. provisional application Ser. No. 62/843,059, which was filed May 3, 2019.

### FIELD

A field of the invention is ophthalmic devices. The invention provides devices and systems for remote ophthalmic screening and diagnostics.

### BACKGROUND

Ophthalmic devices permit a professional to conduct eye examinations. State of the art devices are built primarily for office examinations by professionals. Remote medicine holds great potential to reduce costs, improve patient care and shift focus to substantive examination and professional-patient interaction.

With current ophthalmology practices, patients are normally examined by an ophthalmologist or other medical professional at a professional office, such as an ophthalmologist's office or a clinic within or outside a hospital. A substantial majority of ophthalmic consultations include screening for various eye diseases or conditions. Monitoring is also employed during treatment. For example, eye diseases or injuries managed by topical or systemic medication or surgery often demand regular follow-up examinations to avoid and/or detect complications.

The on-site ophthalmic consultations are expensive, time consuming and laborious for both medical professionals and patients. Consultations are therefore reduced to the minimum required to reasonably manage the risk of ophthalmic disease. Documented observations are therefore purposely limited to maximize economic efficacy and to satisfy demands of third-party payees, such as insurances companies, rather than to maximize screening and follow-up data.

### SUMMARY OF THE INVENTION

Preferred embodiments of the invention provide ophthalmic devices that can be used in traditional professional office settings or can be used for the practice of remote examination by a professional and/or assessment algorithm. Data may be uploaded via internet connection or can be stored and later uploaded or otherwise provided for analysis to a system, such as machine learning system, or to a professional. Preferred devices enable a patient to self-align or adjust device themselves without the help of a professional, collect and store data and can transmit data when a connection is available to a professional or an analysis system, such as a machine learning system, for evaluation. With machine learning, initial analysis can be provided by software in a preferred system for confirmation or evaluation by a professional and can flag information for a patient if data reveals an urgent condition.

A preferred embodiment provides an ophthalmic device including a hand-held housing with an eye facing side, a slit beam lamp associated with the housing for directing a beam of light onto and into the eye of a patient having an eye placed up to the eye facing side, a sensor to image the eye of the patient, and a data interface for providing image data of the camera to ophthalmic analysis software or an ophthalmic professional.

Another ophthalmic device includes a hand-held housing that defines contours on its eye facing side contoured to match a patient face and includes two-eye cups for a patient to align the patient's eyes and optics for directing a beam of light from a slit beam source in the housing to one eye and image data from a display in the housing to another eye.

Another ophthalmic device includes housing enclosing magnets to attach the device to a smartphone, a two way mirror positioned by the housing to align with a camera of the smartphone, and a slit light source within the housing and optics to direct a slit light beam onto and into a patient's eye when the patient is focusing on a reflection of the patient's eye in the two way mirror.

### BRIEF DESCRIPTION OF THE DRAWINGS

FIG. 1 a schematic cross-sectional view of a hand-held screening device for remote ophthalmic diagnostics according to a preferred embodiment of the invention;

FIG. 2 a schematic cross-sectional view of a hand-held screening device consistent with FIG. 1 and having additional slit-beam light sources;

FIG. 3 a schematic cross-sectional view of a hand-held screening device consistent with FIG. 1 and having slit-beam light sources with multiple elevation angles; and

FIG. 4A a schematic cross-sectional view of a hand-held screening device consistent with FIG. 1 and having and having a preferred eye cup; FIG. 4 B shows a variation of the FIG. 4A device that includes optics for reading intraocular pressure assisted by an implanted intraocular sensor 23;

FIG. 5 a schematic cross-sectional view of a hand-held screening device consistent with FIG. 1 and having intraocular pressure measurement;

FIG. 6 a schematic cross-sectional view of a hand-held screening device consistent with FIG. 1 and having multiple chambers for multiple diagnostic tests;

FIG. 7 illustrates steps taken by a patient using the FIGS. 1-6 hand-held screening devices for remote ophthalmic diagnostics

FIGS. 8A-8B are perspective view of a hand-held screening device for remote ophthalmic diagnostics that attaches to and interfaces with a smartphone according to a preferred embodiment of the invention; FIG. 8C is an exploded view of the FIGS. 8A-8B device, and FIG. 8D is schematic diagram of the FIGS. 8A-8C device; FIGS. 8E and 8F are schematic diagrams of a variation of the FIGS. 8A-8D device; and

FIGS. 9A-9C are perspective views of another preferred hand-held screening device for remote ophthalmic diagnostics that attaches to and interfaces with a smartphone.

### DETAILED DESCRIPTION OF THE PREFERRED EMBODIMENTS

Preferred embodiments of the invention provide ophthalmic devices that can be used in traditional professional office settings or can be used for the practice of remote examination by a professional and/or assessment algorithm. Data may be uploaded via internet connection or can be stored and later uploaded or otherwise provided for analysis to a system, such as machine learning system, or to a professional. A preferred device provides remote ophthalmic examinations and includes one, plural or all of the following capabilities: slit lamp examinations, visual acuity examinations, fundoscopy, and eye pressure assessment. Preferred devices enable a patient to self-align or adjust device themselves without the help of a professional, collect and store data and can transmit data when a connection is available to a professional or an analysis system, such as a machine learning system, for evaluation. With machine learning, initial analysis can be provided by software in a preferred system for confirmation or evaluation by a professional and can flag information for a patient if data reveals an urgent condition.

In a preferred device, a slit lamp feature allows the patient to turn on the slit light source and shine a slit onto the eye cutting into the anterior chamber, much like the standard slit lamp. A fundus feature allows the patient to direct the light source to the posterior chamber of the eye and view the retina, namely, the fundus, macula, posterior pole, and optic nerve regions. The visual acuity feature allows the patient to take a visual acuity examination and determine the acuity score. A pressure feature allows the patient to take intraocular pressure measurements. A preferred device is internet enabled and would allow for ophthalmologists or physicians to remotely administer eye exams. Benefits of preferred devices include: (1) the device enables remote examination (2) provides the ability to self-focus, adjust, and examine the eye without a skilled secondary person (3) includes modularity and integrative capabilities to tune the examination choices to the patient needs. A patient or physician can administer any of the previously described examinations in any setting. The patient is instructed to look into an eyepiece of the device. A correct light source projection or display will appear and the examination will be performed. A physician can view remotely live, or images can be captured to be reviewed at a later time. Preferred devices provide a “clinic in a box” instrument which any person can use to perform examinations for monitoring purposes of their eye condition. It can also be used as a pocket tool for physicians to perform quick examinations both in person and remotely on patients.

Preferred systems and method provide remote ophthalmic screening and follow-up that is inexpensive and convenient. This can enhance medical outcomes while also satisfying economic concerns with examination while allowing patients to self-perform remote screening. This enables more frequent measurements, for example daily, multiple times per day, or even continuously, generating far more data points than available with a clinic-based set-up. Data can be provided to medical professionals, who then have access to more complete patient data without the expense and inconvenience of requiring patient visits and on-site data acquisition.

Preferred devices of the invention permit ophthalmic reading and imaging techniques to be carried out by a patient consistently and reliably and provide data that is comparable, or even more comprehensive and accurate, than data obtained by an ophthalmic professional trained to use standard clinical instruments. Devices and systems of the invention provide for measurements including, for example visual acuity, intraocular pressure measurement, biomicroscopy, and evaluation of the posterior pole of the eye.

A preferred embodiment device is a hand-held remote ophthalmic screening device. The device includes a substantially closed chamber provided with an eye facing side. A concave mirror is in the chamber and positioned to reflect an image of the eye along an optical path in the chamber back towards the eye facing side, thereby allowing a subject to focus on his or her own eye. The device includes a slit light source positioned to shine a slit onto and into the eye of a patient, a sensor to image portions of the eye illuminated by the slit light source, and memory for storing sensed images. The chamber and mirror are configured such that a patient can position the eye facing side of the device is located in front of a subject's eye, thereby creating a close-up image of the subject's eye within the focal distance of the eye, hence allowing for an ophthalmic screening process, for example through biomicroscopy, Preferably, the device has Internet connectivity, thereby facilitating Internet-based imaging, data transfer and communication techniques, through which the status of the eye can be documented without the physical presence of both a doctor and patient in the same room. Preferred devices of the invention also can include data input, such as voice input to record verbal input from a patient, in addition to and in association with data concerning visual acuity, intraocular pressure measurements, bio-microscopy, and evaluation of the posterior segment and in particular the posterior pole of the eye.

Internet connectivity can be through a wired or wireless interface with another device, such as a computer, tablet or smartphone. Systems of the invention can include an app on such a device. The app can include a user interface that provides instructions for conducting a particular test, and a confirmation of when the test has been successful. The app can provide for secure data transfer between a patient and a provider with encrypted communications such as used by medical apps and banking apps. Data handling and storage can be according to local regulations about patient privacy. Additional apps, particularly with regard to visual acuity measurement, glasses and contact lens fitting, can be though a provider that supplies glasses and contact lenses to allow fitting/examination to be conducted remotely.

Preferred embodiments of the invention will now be discussed with respect to the drawings. The applications and broader aspects of the invention will be understood by artisans in view of the general knowledge in the art and the description of the experiments that follows.

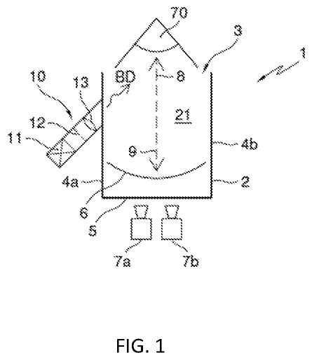

In FIG. 1 A, a hand-held remote ophthalmic diagnostics device is configured to allow a patient to focus on his or her own eye and therefore to consistently and reliably position his eye in front of the device to produce an ‘in-focus image’, enabling subsequent digital imaging and/or measurements. The outer dimensions may vary in size depending on its functionalities and the equipment in its chamber, but it should preferably be portable, preferably not exceeding circa 1-10 kg and more preferably within circa 0.05-1 kg, with outer dimensions preferably not exceeding circa 50×50×50 cm or circa 0.125 m³, and more preferably within circa 10×10×10 cm or circa 0.001 m³. The device 1 may have one or more openings for the subject to look into, with the openings being preferably within circa 50-100 cm² or more preferably within circa 15-25 cm².

The hand-held screening device 1 has a substantially closed chamber 2 provided with an eye facing side 3, sidewalls 4a-b and a bottom wall 5 surrounding an interior 21 of the chamber 2. The shown chamber 2 is substantially box-shaped, but may have another geometry, e.g. a cylinder having a circular cross section. Otherwise, the chamber has a cross section that is polygonal, e.g. as a square. The eye facing side 3 may be completely or partially open, preferably having dimensions exceeding the front dimensions of the human eye. However, in principle, the eye facing side 3 may be closed though optically transparent for performing ophthalmic optical measurements or when the device is not in use.

A concave mirror 6 accommodated in said chamber 2 is positioned to reflect an image of an eye 70, located in front of the eye facing side 3, along an optical path 8, 9 in the chamber 2 back towards the eye facing side 3 of the chamber 2. Then, a subject looking into the chamber, via the eye facing side 3 and into the concave mirror 6, observes his/her own eye 70. In FIG. 1 A, the concave mirror 6 extends in a plane substantially parallel to the eye facing side 3 of the chamber. Then, the image of the eye travels via an optical path 8, 9 that is substantially parallel to the sidewalls 4a-b of the chamber 2. The concave mirror 6 preferably has a focal length in a range from circa 1 cm to circa 1 m, preferably in a range from circa 5 cm to circa 20 cm. Preferably, a distance between the eye facing side 3 and the concave mirror 6 is in a range between circa 1 cm and circa 1 m, preferably in a range between circa 2 cm and circa 10 cm, more preferably in a range between circa 4 cm and circa 6 cm, e.g. circa 5 cm

While FIG. 1 A shows a single concave mirror 6, multiple concave mirrors can be utilized to permit a patient to focus the device using the best image of his or her own eye using any of multiple projections within the device 1, to account for differences in visual acuity to allow patients to focus on the image. The device 1 can be construed such that if the image of his own eye is in focus for the patient, the image is also in focus for all other readings and imaging elements, for example a slit-beam, a visual acuity display, etc. Camera software can use conventional lens system ‘self-focusing’ procedures, and any camera should also be positioned within the depth of focus range of the mirror system.

The concave mirror 6 should preferably be circa 0.5-12 cm in diameter and more preferably circa 2-5 cm in diameter. The mirror 6 should be positioned within the chamber 2 of the device 1 so that it clearly reflects the subject's own eye, ideally parallel to the eye facing side 3 of the device (or physically oriented at a different angle if multiple mirrors are used, to produce a similar image as with a parallel orientation). The higher the power of the concave mirror, expressed in diopter, the power being the reciprocal of the focal length, the more depth of focus of the object, which is useful for the evaluation of the human eye, since tissue structures of the eye are imaged at larger distances relatively to each other, and can therefore be better identified, imaged, studied and measured.

The device 1 is preferably used at room temperature or within circa 0-40 degrees Celsius, and more preferably within circa 18-25 degrees Celsius, to avoid condensation over the mirror(s) and/or other elements like any internal optical elements or parts thereof. The concave mirror 6 preferably is a two-way mirror, as is the case in FIG. 1, thereby allowing the positioning of active optical units such as a camera and/or display screen in the plane of or behind the mirror 6. Alternatively, the mirror may contain a small hole to accommodate the camera, that may be positioned centrally or off-axis, for example in the blind spot of the subject's eye. In this respect it is noted that a camera(s) positioned in the plane of the concave mirror may not interfere with its image formation because they are within the focal distance of the eye). FIG. 1 includes two cameras 7a-b arranged next to each other and behind the concave mirrors 6, opposite to the eye facing side 3. The two cameras are arranged for stereo-imaging of the subject's eye.

In FIG. 1, a single slit-beam lamp 10 is shown and includes a light source 11, a shield having a slit 12 and a convex lens 13. The light beam is directed in a beam direction BD towards the eye facing side 3 for projecting a slit-beam onto and into the subject's eye 70 in front of the eye facing side 3. The lamp 10 is preferably positioned out of the line of sight of a subject as shown in FIG. 1, i.e., projected at an angle into the eye away from a location outside the field of view when a subject is using the device 1.

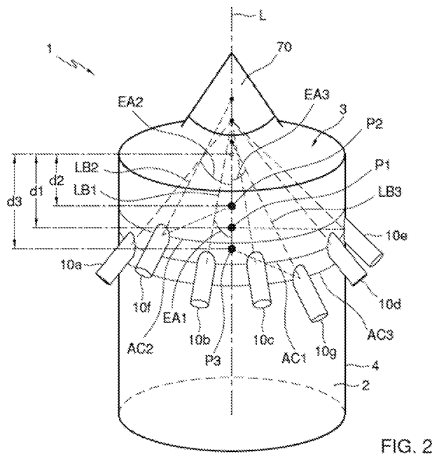

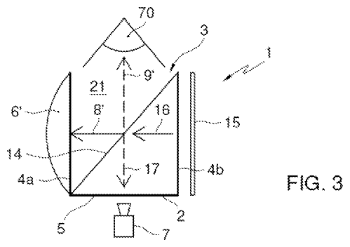

As shown in FIG. 2, the device 1 may optionally include a multiple number of slit-beam lamps 10a-10g arranged on the sidewalls 4 of the chamber, preferably mainly uniformly distributed in a circular direction around a longitudinal axis L of the chamber 2 oriented transversely to the eye facing side 3 for illuminating the eye 70 from multiple and different circumferential locations, e.g. at various angles, e.g. ranging from 0 to 360 degrees around the longitudinal axis L. The sidewalls 4 generally extend in a transverse direction relative to the eye facing side 3 and substantially parallel to the longitudinal axis L. Further, slit-beam lamps 10a-10g can be located at different offsets to the eye facing side 3, along the longitudinal axis L, to illuminate the eye 70 at distinct elevational directions with respect to the longitudinal axis L to allow for illumination of superficial or deeper intraocular structures. In a preferred embodiment, the slit-beam lamps 10 are arranged on an arc that is rotationally symmetric relative to the longitudinal axis L, e.g. on a single circular contour or semi-circle contour enabling illumination across the eye. Then, the lamps 10 illuminate the eye 70 at the same elevational direction, but from different circular positions around the longitudinal axis L. In another embodiment, the slit-beam lamps 10 are arranged on a section running from a first offset position to a second offset position, mainly parallel to the longitudinal axis L, preferably having a specific rotational orientation with respect to the longitudinal axis L. Then, the lamps 10 illuminate the eye at different elevational directions relative to the longitudinal axis for illumination of superficial and deeper intraocular structures. In yet another embodiment, a first set of slit-beam lamps 10 is arranged rotationally symmetric relative to the longitudinal axis L, on a first circular contour, while a second set of slit-beam lamps 10 is arranged rotationally symmetric relative to the longitudinal axis L, on a second circular contour, closer to or more remote to the eye 70 than the first circular contour, then enabling both circumferential and elevational variation of illuminating beams. The number of lamps can be designed such that the device meets user-specified criteria, e.g. 2, 4, 6, 8, 12 or more lamps.

In FIG. 2, a first set of slit-beam lamps 10a-10e is arranged on a first arc contour AC1 on the sidewall 4 rotationally symmetric relative to the longitudinal axis L of the chamber 2, at a first projected offset position P1 having an offset distance d1 along the longitudinal axis L to the eye facing side 3. A second set of slit-beam lamps (as an example one lamp 10f is shown) is arranged on a second arc contour AC2 on the sidewall 4, concentric to the first arc contour AC1, at a second projected position P2 on the longitudinal axis L having an offset distance d2 along the longitudinal axis L to the eye facing side 3, closer to the eye facing side 3 than the first set of slit-beam lamps 10a-e. A third set of slit-beam lamps (as an example one lamp 10g is shown) is arranged on a third arc contour AC3 on the sidewall 4, concentric to the first and second arc contour AC2, AC3, at a third projected position P3 on the longitudinal axis L having an offset distance d3 along the longitudinal axis L to the eye facing side 3, more remote than the first and second set of slit-beam lamps 10a-f.

The first set of slit-beam lamps 10a-e generate a light beam LB1 having a first elevation angle EA1 relative to the longitudinal axis L, while the second set of slit-beam lamps 10f generate a light beam LB2 having a second elevation angle EA2 relative to the longitudinal axis L, more or less the same as the first elevation angle EA1 for illuminating more remote internal structures of the eye 70. The third set of slit-beam lamps 10g generate a light beam LB3 having a third elevation angle EA3 relative to the longitudinal axis L, also more or less the same as the first and second elevation angle EA1, EA2, for illuminating more superficial internal structures of the eye 70.

The device should generate a slit-beam that can focus on various structures of the eye, irrespective of the refractive error of the eye. An examination is better without aids to correct for the refractive error of the eye i.e. glasses or contact lenses, and the distance relative to the mirror and therefore the eye facing side, will vary with the refractive error if the subject is focusing his eye on the concave mirror. This issue is addressed in preferred embodiments, with a system including the convex lens 13 used to focus the slit-beam having a slightly larger depth of focus than the variation in distance from the concave mirror dictated by a normal range of refractive error (e.g., +12D to −12D). Alternatively, multiple slit-beams may be used to correct for the discrepancy in positioning of the subject's eye, whereby each of these slit-beams is focused to correct for a refractive error range, each preferably within 3-6 diopters of refractive error and more preferably with 1-3 diopters. Instead of an adjustable, rotational slit-lamp, multiple slit-beam sources may be used that are controlled to project a slit-beam sequence (e.g. at 0.5-50 Hz), e.g. for generating a circumferential and/or elevational slit beam sequence, so that the observer experiences an image formation simulating rotational slit-lamp arm as well as longitudinal and side-ways movements on a conventional slit-lamp. A multiple slit-beam set-up in both longitudinal, towards or away from the eye facing side 3, and circumferential directions around the longitudinal axis L, should allow for imaging the tissue structures across the subject's eye as well as superficial to deeper inside that same eye.

The slit-beam(s) may be electronically or mechanically adjusted in height and color. The slit-beam(s) should preferably have a yellow-whitish color, preferably in the range of circa 2700K to 3200K color temperature, but its color may be electronically or mechanically changed to blue or yellow, to allow special examination techniques such as tear film evaluation with cobalt blue filter or yellow barrier filter.

FIG. 3 illustrates an alternative configuration of the device shown in FIG. 1. A mirror 14 is located in the chamber 2 at a tilted orientation with respect to the eye facing side 3 and in the optical path 8′, 9′ between the eye facing side 3 and the concave mirror 6′ now located along a sidewall 4a of the chamber 2. Then, the image is reflected by the mirror 14 towards the concave mirror 6′, back to the mirror 14 and subsequently back to the eye 70. In FIG. 3, the mirror 14 is tilted 45° relative to the eye facing side 3 and the concave mirror element 6′ is oriented transverse relative to said eye facing side 3. However, alternative orientations can be implemented such that the image travels from the eye via the mirror 14 towards the concave mirror 6′ and back to the eye 70 via the mirror 14. Further, in FIG. 3, the mirror 14 is mainly flat and two-way, i.e. a beam splitter, the latter allowing active optical units such as camera and display to be located behind said mirror 14. In this it is noted that in another variation, the mirror 6′ can be flat while and the mirror 14 concave. Further, additional mirrors can be included in the optical path from and towards the eye 70.

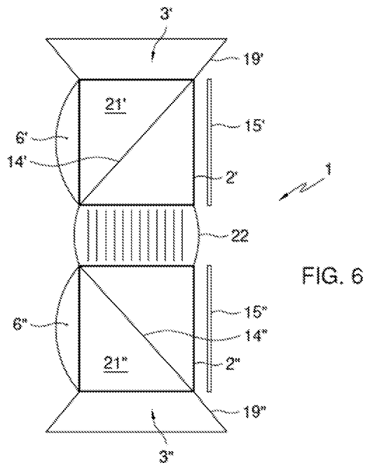

FIG. 3 shows a display element or screen 15 on a sidewall 4b opposite to the concave mirror 6′, behind the two-way mirror 14. Further, a camera 7 is located on the bottom side 5 of the chamber 2. Then, the camera 7 is oriented transverse with respect to the display 15. In principle, one or more separate display screens and/or cameras can be applied. Also optics of a camera can be used as a display. A projection can be established via an optical path 16 from the display screen 15 towards and onto the two-way mirror 14, to create an image behind that mirror, in which case the mirror has to be a two-way mirror in both directions. Similarly, an image traveling from the two-way mirror 14 via another optical path 17 towards the camera unit 7 can be captured. Alternatively, a projected image may be created at a different portion of the device 1, for example by providing multiple chambers 2 as explained in more detail referring to FIG. 6. The position of the sensors for taking measurements is generally in the vicinity of the cameras, unless a more suitable position is available or indicated. The distance between a central portion of the tilted mirror element 14 and the concave mirror element 6′, the camera unit 7 and the display element 15 is preferably the same or substantially or proportionally the same. In the latter case, the concave mirror may be positioned in an asymmetric manner providing a larger distance to the display and/or camera.

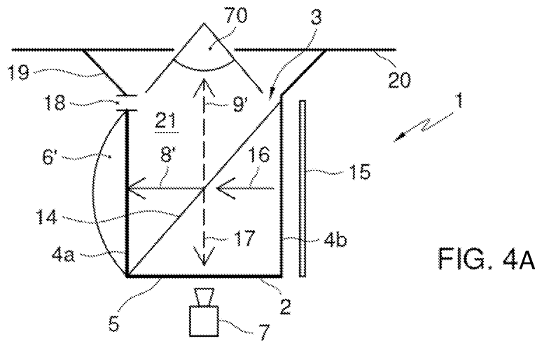

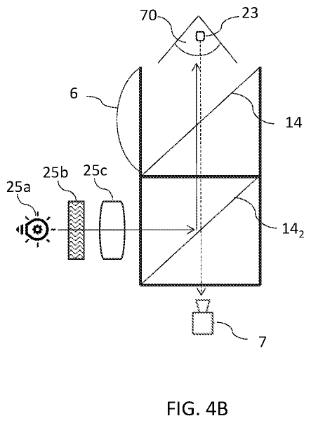

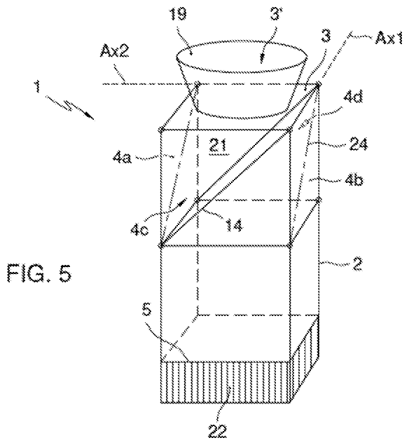

FIG. 4A illustrates a preferred annular shaped non-transparent or two-way cup or suction cup 19 attached on the eye facing side 3 of the chamber 2 and extending away from chamber 2. After placing the cup 19 on a periocular and/or facial skin 20 of the subject, the interior 21 of the chamber 2 is completely closed by the exterior surface of the subject's eye, and a pressure in the chamber interior 21 can set, independently from the atmospheric pressure. The device 1 further comprises a pressurizer such as a balloon or an automated pressure device for pressurizing the chamber interior 21 for facilitating intraocular pressure measurements as explained in more detail with respect to FIG. 5. Generally, the chamber 2 may include an inflating port 18 connected or connectable to the pressurizer, the inflating port being provided with a one-way valve allowing air to flow into the chamber while blocking air to flow outwardly form the chamber. Similarly, the suction cup 19 may include a deflating port connected or connectable to a de-pressurizer, the deflating port being provided with a one-way valve allowing air to flow outwardly from the suction cup while blocking air to flow inwardly, into the suction cup 19. The suction cup 19 can be provided with a double wall structure defining an interior volume that can be depressurized for sucking the cup 19 against the periocular and/or facial skin 20 of the subject. Alternatively, the material of the cup is flexible enough to create contact the periocular and/or facial skin 20 of the subject and creating an airtight seal.

Preferably, the illumination level within the device can be controlled by the non-transparent suction cup 19 positioned onto the periocular skin, so that no ambient light interferes with the imaging of the subject's eye. The ‘ambient light’ within the device can be controlled by providing a diffuse light source arranged in the chamber 2. Alternatively, the suction cup 19 may be construed from a two-way material for light, so that all outside ambient light is blocked while the subject's eye position can be visualized by an observer, e.g. an instructor explaining the use of the device to a patient.

Static letter charts are routinely used in the ophthalmic practice, with smaller letter sizes (smaller angle of resolution) representing higher visual acuities. In the device 1, a similar principle may be used to expand on the method both doctors and patients are familiar with. However, the method may be improved in several ways. First, the chart shown on the display element 15 may be dynamic through displaying a variable letter size, but with different letters, to prevent visual acuity level bias through recall. Second, the chart may show various shades of contrast and color, to enable simultaneous contrast sensibility and color vision readings. Third, the projector may display the letter size based on an average visual acuity level measured by the device with a specific patient on previous occasions.

FIG. 4 B shows a variation of the FIG. 4A device that includes optics for reading intraocular pressure assisted by an intraocular sensor 23. The sensing methods and the sensor 23 can be conducted as in Phan et al., Optical Intraocular Pressure Measurement System for Glaucoma Management, 2017 IEEE Healthcare Innovation Point-of-Care Technologies (HI-POCT) Conference; and as in Phan et al., “A Wireless Handheld Pressure Measurement System for In Vivo Monitoring of Intraocular Pressure in Rabbits,” IEEE Transactions on Biomedical Engineering (Jun. 24, 2019). FIG. 4B omits some features of FIG. 4A, such as the display element 15, slit light sources, and the optical paths associated with the convex lens for a patient to self-align by viewing an image of their own eye, for clarity of illustration of features added and discussed in FIG. 4B. An interference light source 25a with filter 25b, and lens 25c directs light via an additional beam splitter 142 to interact with the implanted sensor 23. Pressure on the sensor 23 affects an interference pattern, which is detected via the camera and can be analyzed as in the cited publications. Specifically, interference fringes are formed when the light from the interference light source 25a interacts with the implanted sensor 23.

FIG. 5 illustrates a preferred balloon 22 for pressurizing the chamber interior 21. As an alternative, an electronically controlled pressurizing element can be used. The device 1 further comprises an additional mirror 24 located in the chamber 2 in a tilted orientation with respect to the eye facing side 3 defining an additional optical path between the eye facing side 3 and an active optical unit, wherein tilting axes AX1, AX2 of the mirror 14 and the additional mirror 24, respectively, are transverse with respect to each other. Generally, the tilting axes of mirrors 14 and 24 do not coincide, thereby creating separate optical paths. Then, multiple active optical units such as cameras and/or displays can be made visible, simultaneously, to the user.

Conventional intraocular pressure measurement devices use contact, semi-contact or intraocular methods. Application tonometry measures the degree of flattening induced by a circular plastic object touching the cornea. Puff tonometry is effective through measuring corneal deformation induced by an ‘air puff’. If automated, all these methods are prone to error and failure because they require considerable skill and training to produce reliable and consistent readings. Furthermore, all methods—even when calibrated—remain an estimate rather than a true reflection of the pressure inside the eye. An accurate intraocular pressure measurement may be obtained with various intraocular devices, but all of these solutions first require a surgical procedure to implant (part of) such a device inside the eye. Some devices and method of the invention avoid any implanted intraocular devices and can still measure intraocular pressure. However, devices of the invention, for example the preferred FIG. 4B device can also measure intraocular pressure with the assistance of an intraocular implant.

A new model using non-contact measurement is preferred by preferred embodiments of the present invention. Devices of the invention can rely on hydration status, as it is has been found that the hydration status of the cornea, crystalline lens and retina varies with the intraocular pressure, rendering specific changes in thickness, diameter, transparency, diffraction and polarization. Devices can rely on blood flow detection, as it has been found that multiple anatomical structures show detectable changes that vary with the intraocular pressure level, for example the arterial and venous blood flow (and the ratio between them), the actual blood volume within the ocular structures like the iris, ciliary body, retina uvea and choroid (squeezing empty effect with higher pressures), muscle contraction and relaxation status and times thereof.

Techniques for measurements using the present models can include projection of patterns, e.g., concentric rings, onto the cornea, crystalline lens or retina, to allow for contour variations that can indicate the intraocular pressure level through a change in (color) diffraction patterns and higher order aberrations. These methods may be combined with corneal and crystalline lens transparency measurements by densitometry (backward scatter) and stray light measurements (forward scatter), pachymetry and lenticular thickness measurements, as well as corneal, crystalline lens and retinal polarization measurements, to identify threshold values indicating pathology. Sensitivity and specificity can be improved with Doppler flow readings, which can measure the (change in) arterial and venous perfusion throughout the limbal area, the iris, the ciliary body, the retina, the uvea and the choroid, as well as the main vessels entering the eye through the optic disc. In particular, the ratio between arterial and venous flow was found to be indicative of the (change in) intraocular pressure, since the arterial and venous flow show an asymmetrical reduction with increasing intraocular pressure levels. Furthermore, the blood volume content of various structures can be measured with infrared light, ultrasound and other imaging methods. Additionally, testing can be improved with a pressure-chamber to equalize the intraocular pressure. After positioning the suction cup of the device air-tight onto the periocular and/or facial skin 20, the pressure can be easily increased inside the device, by compressing a balloon on the outer side of the device or by using an automated pressurizing element, that is coupled via a pressure valve with the interior pressure chamber. At the point at which the pressure inside the pressure chamber just exceeds the intraocular pressure, the corneal contour of the subject's eye will start to change, most commonly by central indentation, which is registered by diffraction, polarization or refractive power changes, as described above. The suction cup can include a second balloon or automated system to create a negative suction pressure over a double walled suction cup relative to atmospheric conditions, e.g. an underpressure.

FIG. 6 illustrates a variation of the preferred device with a plurality of substantially closed chambers 2′, 2″ each provided with an eye facing side 3′, 3″, and optical components accommodated in said chamber for performing respective measurement on the subject's eye. Two-way or two-way mirrors 14′, 14″ are located in a tilted orientation with respect to the eye facing sides 3′, 3″ such as to generate an optical path from the respect eye facing sides, via the mirrors 14′, 14″ towards respective concave mirrors 6′, 6″ located on a sidewall of the chambers 2′, 2″. Multiple chambers allow, for example, a chamber for visual acuity measurements, another chamber for bio-microscopy and posterior pole imaging, and yet a further chamber for intraocular pressure evaluations.

Preferred devices of the invention include a control unit or processor for electronically operating the device 1, more preferably also including a local and remote user interface for selecting operations. Further, the device 1 may have Internet connectivity. All measurements and imaging data obtained can then be digitally transferred to a remote observer location and stored into a database to support the algorithms for detection of anatomical, functional or secondary changes from the data points on average with an increasingly narrow margin to detect relevant ophthalmic deviations and/or pathology. Hence, the system allows the remote observer to examine the subject's eye through real-time or temporally stored images and measurements, supported by multiple data point analysis of measurements performed since the last or any other prior examination or evaluation.

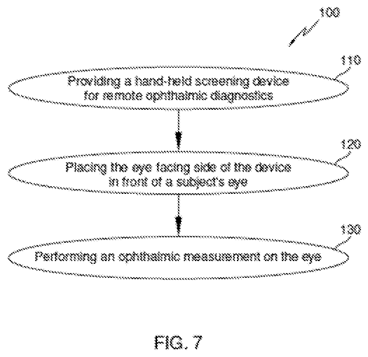

FIG. 7 illustrates steps of a method according to the invention. The method is used for performing screening for remote ophthalmic diagnostics. A step of providing 110 a hand-held screening device to a user is the initial step, and the devices is consistent with FIGS. 1-6. A patient places the patient's eye 120 the eye facing side of the device in front of a subject's eye. The control unit then conducts 130 an ophthalmic measurement on the eye.

The step of performing an ophthalmic measurement on the eye can be performed using dedicated hardware structures, such as FPGA and/or ASIC components. Otherwise, the method can at least partially be performed using a computer program product comprising instructions for causing a processor of a computer system to perform the above described steps. A number of steps can in principle be performed on a single control unit or processor. However, it is noted that respective ophthalmic measurements can be performed on a separate control unit or processor. As an example, a sub-step of driving a display can be carried out by a first processor while a sub-step of controlling operation of a camera unit can be carried out on a second processor.

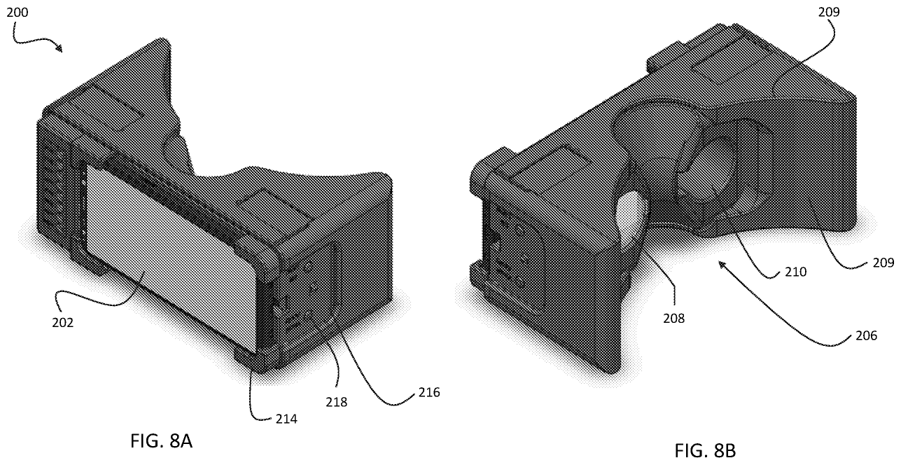

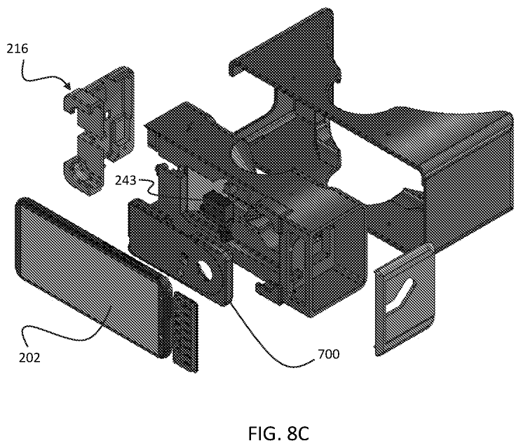

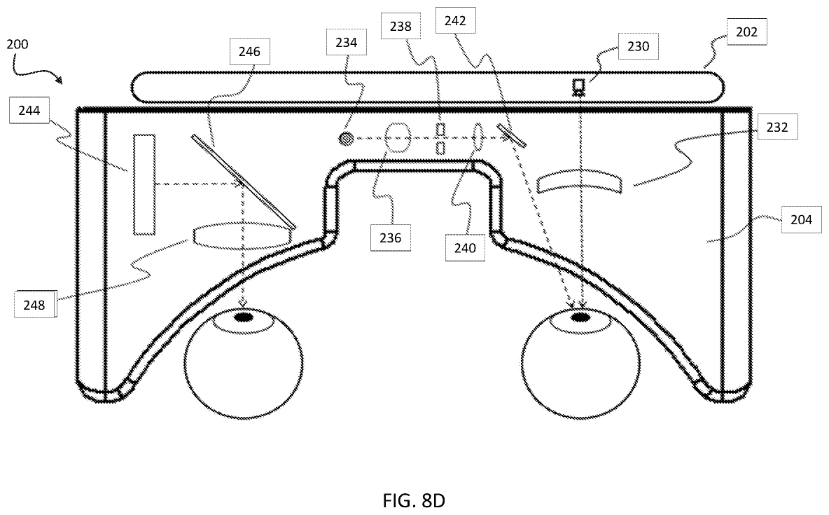

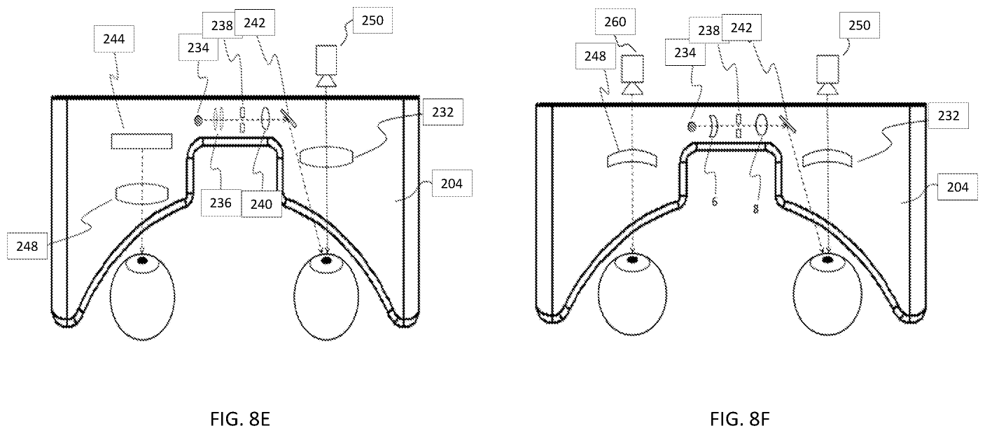

FIGS. 8A and 8B are perspective views of a preferred embodiment ophthalmic device 200 that leverages a smart phone 202 and includes a hand-held housing 204 defining an eye facing side 206. The eye facing side 206 is shaped and configured with contours 209 to closely fit on a patient's face, with separate left and right eye cup portions 208 and 210. A holder 214 accepts and holds the smart phone 202, preferably at the corners. The holder 214 can slide open to accept the phone 202 and preferably can lock in multiple positions to accommodate different sizes of smart phones. A user interface 216 includes buttons 218 for activating the device and conducting tests. FIG. 8C is a schematic diagram of the device 200 with the smart phone 202 attached. The device 200 leverages a camera 230 of the smart phone 202 to image eye structures. A two-way concave mirror 232 allows the patient to position the device and focus on his or her own eye. Additionally, the two-way concave mirror 232 allows the camera 230 to image the patient's eye. The slit lamp includes a light source 234 that emits a beam that passes through a collector lens 236 and slit 238. A projection lens 240 projects a slit beam via angled mirror 242 onto and into a patient's eye. The mirror 242 can be mounted on a servo or motor 243 (see FIG. 8C) and the mirror's angle can be modulated so that the slit beam is projected at various locations across the patient's eye. An LCD screen 244 can present stimulus to a patient's eye via another angled mirror 246 and a lens 248. The lens 248, preferably is a biconvex with diopter circa 20D-40D, is integrated to allow patients to focus the display on LCD screen 244 regardless if they are far-sighted, near-sighted, or normal. A patient simply turns the device 200 on and presses on a button corresponding to either a slit lamp examination or visual acuity examination to start. The patient then positions their eyes in front of left and right eye cup portions 208 and 210 and begins the examination. A physician can remotely connect to the device camera through a mobile application and review the patient eye remotely without the need of physical presence.

With respect to FIG. 8D, the left side of the device 200 can conduct a visual acuity test on a patient's left eye, and the right side of the device can conduct slit light examination and imaging of the patient's right eye. The device 200 can be flipped, as it is shaped and contoured to allow the patient to switch testing on the eyes, such that the visual acuity portion of the device can be used with the right eye and the slit light testing with the left eye.

FIGS. 8E and 8F show variations of the FIG. 8A device that don't use a smart phone. In the FIG. 8E device, a built-in camera 250 is included (shown separately from the housing for clarity but can be included in the housing itself). Another variation is that the LCD screen 244 is in line with a patient's eye, so the angled mirror 246 is omitted. Other features are labeled as in FIG. 8C. FIG. 8F modifies FIG. 8E by including a projector 260 in place of the LCD screen 244 to provide visual stimulus through the lens 248.

Operations and additional features of the FIGS. 1-6 devices can also be included in the FIGS. 8A-8F devices. For example, while one slit light beam and source is shown in FIGS. 8A-8F, the housing 204 provides room to additional slit light paths to provide additional slit light beam paths onto and into the eye. The housing 204 and eye cup portions 208 and 210 can seal and the housing 204 can include pressurization features discussed above. An app installed on the smart phone or included in a dedicated hardware/firmware in the housing 204 can provide operations including pressure measurement via Doppler flow readings, as discussed above.

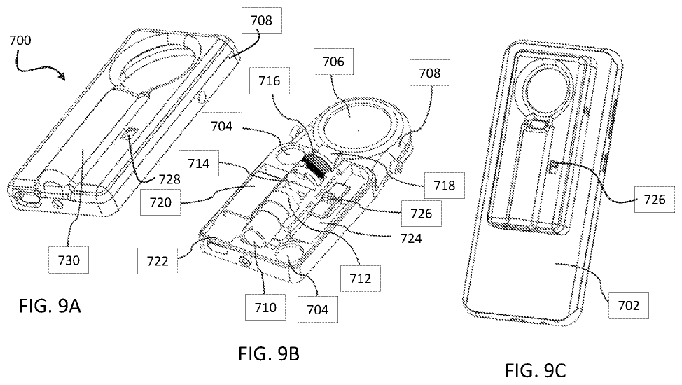

FIGS. 9A-9C show another preferred hand-held ophthalmic device 700 that is configured to attach to a smartphone 702, via magnets 704 as shown attached in FIG. 9C. The device 700 includes a two-way concave mirror 706 that is positioned by a housing 708 to align with a camera of the smartphone 702. A slit lamp source 710, such as an LED generates a light beam that is collected by a collection lens 712, which beam is then converted to as slit by a slit structure 714, projected by a projection lens 716 and an angled (or multiple angled) mirror(s) 718. The mirror(s) 718 are positioned below the two-way mirror 706 outside of a field of vision such that the slit light beam can pass onto an into a patient's eye while the patient is focusing on the reflection of the patient's eye in the two-way mirror 706. A battery powers the lamp source 710, and can be charged through a charging port 722. A circuit board 724 controls the on and off state of the LED. The ophthalmic device 700 operates as a stand-alone device and can be attached to a phone camera. The functionality of device 700 is preferably irrespective of the app on a phone. A switch 726 turns the device 700 on, and is exposed through a hole 728 in a top cover 730 that closes the device 700. The stand-alone slit device 700 can be integrated into a frame, as shown in FIG. 8C, and connected to the other examinations such as fundus, pressure measurement, and visual acuity to create a multi-function device.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. An ophthalmic device comprising a hand-held housing with an eye facing side, a slit beam lamp associated with the housing for directing a beam of light onto and into the eye of a patient having an eye placed up to the eye facing side, and a sensor to image the eye of the patient.

2. The device of claim 1, comprising a data interface for providing image data of the sensor to ophthalmic analysis software or an ophthalmic professional, preferably wherein the data interface is an internet connection and/or a wireless connection.

3-5. (canceled)

6. The device of claim 1, comprising visual acuity means for measuring visual acuity, pressure measurement means for measuring intraocular pressure and/or alignment means for aiding the patient to align the device.

7-8. (canceled)

9. The device of claim 1, wherein the hand-held housing comprises a side that holds and interfaces a smartphone, and an opposite side includes a lens in the eye facing side, and the data interface is through the smartphone.

10. The device of claim 1, wherein the slit beam lamp is configured and positioned to project a slit light into the anterior chamber of the eye and/or into the posterior chamber of the eye.

11. (canceled)

12. The device of claim 1, wherein the hand-held housing comprises a substantially closed chamber defining the eye facing side, the device further comprising a concave optics said chamber to reflect an image of the eye along an optical path in the chamber back towards the eye facing side of the chamber, thereby allowing the patient to focus on the eye.

13. The device of claim 12, wherein a focal length of the concave optics is in a range from circa 1 cm to circa 1 m.

13. The device of claim 12, wherein a focal length of the concave optics is in a range from circa 1 cm to circa 1 m.

14. The device of claim 13, wherein the focal length of the concave optics is in a range from circa 5 cm to circa 20 cm.

15. The device of claim 12, wherein the concave optics provide a plurality of projections of the eye so the patient to focus on a projection matching the patient's visual acuity.

16. The device of claim 1, wherein the sensor comprises a camera and the camera is a self-focusing camera or the sensor comprises a pair of cameras arranged for stereo-imaging of the eye.

17. (canceled)

18. The device of claim 1, wherein the slit beam lamp comprises a plurality of slit beam lamps arranged to generate a plurality of slit light beams at a plurality of circumferential positions and elevational angles with respect to the eye preferably wherein the plurality of slit beam lamps are arranged at a plurality of offset distances from the eye facing side.

19. (canceled)

20. The device of claim 1, wherein the hand-held housing comprises a substantially closed chamber defining the eye facing side, the device further comprising a concave mirror and an angled two-way mirror arranged in the chamber to reflect an image of the eye along an optical path in the chamber back towards the eye facing, thereby allowing the patient to focus on the eye, and to provide the sensor with an image path to the eye.

21-23. (canceled)

24. The device of claim 1, comprising a display for displaying visual stimulus to the eye of the patient, preferably wherein the display is controlled to:
provide a dynamic visual acuity test stimulus with varying characters of varying sizes, or
project a pattern onto the eye that permits detection of contour variations indicative of intraocular pressure level.

25-26. (canceled)

27. The device of claim 24, wherein the display and sensor are controlled to make corneal and crystalline lens transparency measurements by densitometry (backward scatter) and stray light measurements (forward scatter),
make corneal, crystalline lens and retinal polarization measurements,
make Doppler flow readings to measure changes arterial and venous perfusion of one or more eye structures, and/or
make blood volume content measurement of one or more eye structures.

28-30. (canceled)

31. The device of claim 1, wherein the hand-held housing defines contours on its eye facing side contoured to match a patient face and includes two-eye cups for a patient to align the patient's eyes and optics for directing the beam of light to one eye and image data from a display to another eye, preferably wherein the image sensor is a camera of the smartphone, or wherein the image sensor comprises a camera built-in the hand-held housing.

32-35. (canceled)

36. An ophthalmic device comprising a hand-held housing that defines contours on its eye facing side contoured to match a patient face and includes two-eye cups for a patient to align the patient's eyes and optics for directing a beam of light from a slit beam source in the housing to one eye and image data from a display in the housing to another eye.

37. The device of claim 36, wherein the device is configured to be flipped so a patient can switch eyes that receive the beam of light and the image data.

38. The device of claim 36, comprising a smartphone holder on an opposite side from the eye facing side, preferably wherein the image sensor is a camera of the smartphone, or wherein the image sensor comprises a camera built-in the hand-held housing.

39. An ophthalmic device comprising housing enclosing magnets to attach the device to a smartphone, a two way mirror positioned by the housing to align with a camera of the smartphone, and a slit light source within the housing and optics to direct a slit light beam onto an into a patient's eye when the patient is focusing on a reflection of the patient's eye in the two way mirror.

40. The device of claim 39, wherein the optics present the slit light source from an angle that is outside the field of vision of the patient when the patient is focusing on a reflection of the patient's eye in the two-way mirror.

41. A system including a device of claim 1 and further comprising software for receiving the data and conducting an ophthalmic measurement using the data, preferably wherein the eye has an implanted sensor, and light from outside is directed at the eye to form interference fringes that can be read out with the system or wherein the eye has an implanted sensor, and the response of the sensor due to pressure changes can be read out with the system.

42-43. (canceled)

44. A system according to claim 41, wherein the response of the sensor is an optical response.

45. Software for processing data from a remote ophthalmic device of claim 1, the software comprising code for receiving the data and conducting an ophthalmic measurement using the data.
